SEQUENCE LISTING (3 sequences)

SEQ ID NO: 1 - DNA, 9098 bp, Sus.scrofa
  exon (827)..(924)
  intron (925)..(5364)
  exon (5365)..(9098)
gagctcctgttgtggctcagtaggttaagaacccagcacagggtctgtgaggattcgggttcaatccttggtctcgatcagtgggttaaggatccgtcattgccgtgagctgtggtgtaggttgcggatgcaagtggatccggtgtggctgtggtgcaggctggcagatgcaactccaattccacccctaacccaggaacttccatatgccgcaggtgcagtcctgaaaagaaaagaaaggaatggaaaagcaaggaaaggaaaggaaaagaaaagaactccagtaatgtaaacatggtgttgttgcaaggagcacagaattccctgactgagaactgaccagggaggagaattctgcgggtggtttggttaccatttcaaaattataataaaaagtgctcagatagttagcatttgatgagtatctgctaaaggtaaacactgtcccaagtcttctcctataacaagtctcactataacctcattgactctcagaaatgactcttcagatttccagcttgcaggtggggaaactaaggcacagagctgcgggagcttagcacaagcccgagcttatgttgacaagatcatctccaacccaacagccttggggagcgaactggcagctttggcagtgctccagcacacacccatgtgtgggtcttcagatcccccagttaagcttcctgaagagccaggtgccagctggagctgggggctcctggctggtgattggctgccatcactggtggttagccctctggggtggtgggggggaccgggaaggacaggggaggctgataaataaagcagccatctgctctgagggatgggagagcacattttgccttctctttggtagcgagtaatatcaaccgaagtgcagacatcctaacactttgcccaggcagcctgctgagcaaggtaacactcatccttttcatgccttgagtcacaatatttatgacatttttatgtttctcattagaagtacttgggctttgttccttgcggggaatgagaggagggagcaagggacgctggcgtacatttgacttttcttgctaatgtacgaaaataggaaaggaaccagaaaccaagttgaggaagacactgtacgttcaaagagaagacggaaaatatctgaattactgtaaataaatgctgcaggcagaggaagtgtttaaggagtgcttcttgagtggatgtgtaaagcaaatcagcagaaggagaggtgtgagactcttgaaaaaaatattgtgctggtccaggtaataagagtaatcaggagcaaacaaattccagggcgggaggaaatgtacttgcccaggtatttgccagggtagtgccaggaatttcctgaattatataggaccaggagaccagtatttctcagtgtgtgacctgtgggccactgaaatacagcaggatcattgaaatgcttgttaaaaaatatgatctctggagttcccgtcgtggcgcagtggttaacgaatccgactaggaaccatgaggttgcgggttcggtccctgcccttgctcagtgggttaacgatccggcgttgccgtgagctgtggtgtaggttgcagacgtggctcggatcccgcgttgctgtggctctggcgtaggccggtggctacagctccgattcgacccctagcctgggaacctccatatgccgcgggagcggcccaagaaatagcaacaacaacaacaacaacaaaagacaaaagacaaaaaaaaaaaaaaaaaaatatgatctctggactccaccccagataaattgaatcagattctctgggacctgacgatctgccttatttttttaaacccaagttccctagtgattctgatacagagcaattttgagaatgccacggtagagccctacacagaggttatgttcttgaacagggttcctgaccaggggctgaagtgcttcaagcaagaagttgcctggaggaagcgtaagaatcttcaggtcatcttgtagatgcctgagacttataaaaggcttttgatactatcattttttgatgatggtgtttggcaaagagtagacaataatatttgcggagtgaatgaatgagggattaagtgggacaggtgtgacaataaaggatcacacctgggcacactgcagtcactcaaactttgtgtgtggaccaagtggaatatgttaagaggagagatgactgaccaagggccagctcggaggtgtgtgtaaggtgtgtgtgtcgggggagcttggcagggaggtggagggggcgctgctgtaactctgccccctgaggttactgatgattcactaacacaaataacagcttctgaccctgcctgacttgtcaaaatgaatccgtgcccttagagagaaggcccctggttgctgtgatggcaagtgatggcaatactcagagggcagagcccgatgggaacagctgagccagcagcaaccaccctgccttgtagagtctggtccactgctgaaaccttctgttagaagagaggaaatcaagcattcacaaaattcctgagtgtcaaaaaaaaaaaaaaaaaaaaaaaaaagaaagaaagaacgaaaatgactacaagataacccataatcctaactttatgtgctgtcaacaggtccctcttttagtcccctttctgcgtgtttaccagaagagaaaggtaagctaagaatggtagtcagatttgcagcttagccaattaaaaaatttttttattatagttgatttatattgttctgccaatttctgccgttaacttagccaaatcttaaatttttctggggggaaactatagaaaagtgaccttggtacatagaaacctaggctttaaaacatttttttcccctaaggtgaatagatcacaaaaattaaatataaatataaagcagttgagtagaaaagcaatctaataaataaatttgatttggatgtcaacgctagtaactgcatggtgatggtactctttccatggtagtatttacacaaatatagtttaggcagattttgatgcaacatttcaggccactgggagtcaggagaggattgggtagggagaggaggcagttctagggatccttccagtatgccttgaggttgactgactcattgtgaaagttcaatatttcatttgattagctctacagatgaccatgcaattattcataagaactcaaaatatcgtctcggaattccagagaactagtgcattagaaaaggcagggaaagagttccctggtagctcaatgggttaaggacccagcattgtctctgttgtggctcaggtcactgctgtgatgtcggttcaatccttggcccagacacagggaaaggaagggctaaagaaacaaaacacatggaaataagttcaaaccagtgggacagaagtgacatccttgccgctgatcctgaatgtaggaacataactgaacaataagcaactttgaggcgttggtcggactgcagttgaaatacttttgaggttaatgaggcctttgaaatggtttggaactgtttagttctcacacagtcctgcatatagaacagtgtaacaggatttccttttcaaccaagacttggaaattggcttttcgtctttatgaaaaacatgaatctctgaagcatcatgcatgcaagatggaacaaacttctgtggttgaaagggatgacctaacaggaagctttgttagcagcagtcaggatcacccttgtggctttcctttcccctcccccaaatcccacccctgggcaaagtgtgttcatcagaggattagtctccataccacaggacaataacaatatcatcaacttggttagttagttaattgaaggggaagcattgtttgatataattggcgggtcaagagcactaacccaggaaaatatggaaaatttagattgtagtttgaaaatacaattgctgtttttccagtcatgctgatactaaggaattttgagaatattgtgctaattgggtctagagacactcaaatggcatttttaaacttttttctacagctctgttgaagcctacaccacaacccaggttgctagggtttcctggaatatataatggccaatgttttttttgaagaagaaaattattactcccacctcttcttattacaaacgatagagctaatgaccagtgcccagtaatgacttttgggatggcctgtgtgaattcgacctgaaactacttgaatttttcatcttaaaagtcctttggagtctctgtacatgcacactgtggtttgttgcaggtttagagagttctgtgggttttgttcctaatggagacacagccttataatttacagcagcgttcactaattaaaattgtaagcataattactatccatgatacttgttattagtgtgcattcataaagctcaaaattcacttcatcctttcaagtagcgagtaattagtttctttggatttgtagctttatcatccctttatgtcccgtttcagagaaataaacaaccaacctcctggcagactgctttaaaaagctggaaatacattgtccagctagtgcaatgagactgatacagcgcggaaaatatgacttttctttggttttagtaaccagtttttccttgcatttattcagcagataactattgagcatctaatgtgtgctagaaagcttgggggagacagagataaatcactcaaagattccgtcctaaaaaacgtccaggttttggtggaaaggaaataatatcgtacatagataatattgtacatcaactatatctcaataaaaaagaataatttttataaaagaaatgattgtacatatatacgtaaaatagaccatggtgatgatgcgtgaacgcagaaacatgtaaatggacaatgccactgaaagaacaggaaatgactctgttttctgtatatgatcagccctaacatagtatattaacataggaagccaaacaattaactttctgaatgctttaataaatgaataggcgttcccattgtggcgcagtggttaacgaatccaactaggaaccatgaggttgtgggttcgatccctgcctttgctcagtgggttaaggagccggtattgccatgatctgtggtgtaggtcgcagatgcggctcaggtcccaagcagctgtggctctggcgtaggccagcggctgcagctcccctagcctgggaacctccgtgtgctggttctgaaaaaaggcaaaaagacaaaaaaataaaataaataataaaataaaataaaaaataaatgaataattgaatgtactaattggtattccttccaagttgcatcagtgggttatttacattttttttcctaacctgacttgtccttattgctcccaggtacctcagccagcatggctgtctctttgccacccactctgggactcagttccgccccagatgaaatccagcacccccacattaaattttcagaatggaaatttaagctattcagggtgagatcctttgaaaaggcacctgaaaaggctcaaacggaaaagcaggattcctccgaggggaaaccctcgctggagcaatctccagcagtcctggacaagcctggtggtcagaagtcagccctgcctcaaccagcattcaagccccatccaaagtttttaaaggaatcccacgaagatgggaaagcaagagacaaagccatccaccaagccaacctgagacgtctctgccgcatctgtgggaattctttcaacaccactgggcacaagagaaggtatccagtccacgggcctgtggatggtaaaacccaagtccttttacggaagaaggaaaagagggccacgtcctggccagacctcattgccaaagttttccggatcgatgtgaaggcagatgttgactcgatccaccccactgagttctgccataactgctggagcttcatgcacaggaagtttagcagcaccccatgtgaggtttactccccaaggaatgcagccatggagtggcacccccacaccctaaactgtgacatctgccacattgcacgtcggggactcaagaggaagagtcagcagccaaacatgcagctcagcaaaaaactcaaaactgtgattgaccgagcgagacaagcccgtcagcgcaagaggagagctcaggccaggatcagcagcaaggaactgatgaagaagatcgccaactgcggtcagatacatcttagccccaagctcctggcagtggacttcccggcgcactttgtgaaatctatctcctgccagatttgtgaacacatcctggccgacccggtggagaccagctgcaagcatgtgttttgcaggatctgcattctcaggtgcctcaaagtcatgggcagcagttgtccctcttgccactatccctgttttcctactgacctggagagtccagtgaagtcttttctgagcatcttgaataccctgatggtgaaatgcccagcaaaggagtgcaacgaggagatcagcttggaaaaatataatcaccatatctcaagccacaaggagtcgaaggagacatttgtgcatattaataaagggggccggccccgccagcatctcctgtccctgacgcggagggctcagaaacaccgtctgagggagctcaagctgcaagtcaaggccttcgccgacaaagaagaaggtggcgatgtgaagtcagtgtgcctgaccttgttcctgctagtgctgagggcgaggaatgagcacagacaagctgacgagctggaggccatcatgcgaggccagggctccggcctgcagcctgctgtttgcttggccatccgcgtcaacaccttcctcagctgcagccagtaccacaagatgtacaggactgtgaaggccatcacgggcaggcagattttccagcctttgcatgcccttcggaatgcggagaaggtccttctgcccggctaccaccccttcgagtggcagccacctctgaagaatgtgtcttccagcacagacgtgggcattattgatgggctgtctggactctcctcctctgtggacgattacccagtggacaccattgccaagcgcttccgctatgactcggctctggtgtccgctctcatggacatggaagaagacatcctggagggtatgagagcccaagaccttgacgactacctgaatggccccttcactgtggtggtgaaggagtcttgtgatgggatgggagacgtgagtgagaagcacggcagtgggccggtcgtgccggaaaaggccgttcggttttccttcacagtcatgaaaatcaccatcgcacacgggtcacagaacgtgaaggtgtttgaggaagccaagcctaactctgaactatgctgcaagcccttgtgcctcatgctggccgacgaatccgaccatgagaccctgacggccatcctgagccctctcattgccgagagggaggccatgaagagcagccagctaatgctggagatgggaggcatcctccggactttcaagttcatcttcaggggcaccggatatgatgagaaactggtccgggaagtggaaggccttgaggcttctggctctgtctacatctgtaccctctgtgatgccacccgcctggaagcctctcaaaatctggtcttccactccataaccagaagccacgcggagaatttggagcgctatgaggtctggcgttccaacccataccatgagacggtggatgaacttcgggaccgggtgaaaggggtctcggccaaacccttcattgagacggtgccttccatagatgccctccactgtgacattggcaatgcagccgagttttacaagattttccagctcgagataggggaggcgtataagaacccccatgcctccaaggaggaaaggaagagatggcaggcgaccttggacaagcacctccgcaagaagatgaatctgaagcccatcatgagaatgaatggcaactttgccaggaagctcatgaccaaagagactgtggaagcagtctgtgagttaattccctccgaggagaggcatgaagctctgagggaactgatggacctttacctgaagatgaagcccgtctggcgatcatcatgccctgctaaagagtgcccggaatccctctgccagtatagtttcaattcacagcgttttgctgagctcctctccaccaagttcaagtacagatatgagggcaaaatcaccaattattttcacaagacactggcccacgtcccggaaattatcgagagggacggctccattggggcatgggctagcgagggaaatgagtctgggaacaagctgttcaggcgcttccgaaaaatgaatgccaggcagtccaagtactatgaaatggaagatgttttgaaacatcactggttgtacacctccaaatacctgcagaagtttatgaatgctcataaagcatttaaaaactcagggtttaccataaacttgcagagaagctcaggggacacattagacctagagaactctccagaatctcaagatttgatggaattttaagcagggaagttgcctaggcattggttttgcaaatgggttttctctgggttgcatggagagcttctcctggcacccttaactcctgggtgtggggcttcaccacccaagtggtggtaggttgatgaggccagagatggtgaacccatgtcgggaataggtaactggtgagccgattgcttgagctgtttagggagttcagaaaagcaataggagaaaccagttatttgaaagctccatagctgagaacaggggtaactgcaggggaccagaggtgaacaaagatgtgtggagaggattgagtgatgccaaagtcaaagccaaggttgtcaaaggacagccagtgaagccagaaaaggaatttgacttgtggtttccattccccctgcccaagttattctagtttatactgagaggtgtttttcatatcacttttgtagaattttttattttataaatttcacatattttggcttattttacaagatttaaaaaaggttttcaagaattcccgctgtggcacaatgtggcatcttgggagtgctgggacacaggttccatccccagcctggcgtggtgggttaaggatcc

SEQ ID NO: 2 - DNA, 3831 bp, Sus.scrofa
  CDS (113)..(3244)
gggatgggagagcacattttgccttctctttggtagcgagtaatatcaaccgaagtgcagacatcctaacactttgcccaggcagcctgctgagcaaggtacctcagccagcatggctgtctctttgccacccactctgggactcagttccgccccagatgaaatccagcacccccacattaaattttcagaatggaaatttaagctattcagggtgagatcctttgaaaaggcacctgaaaaggctcaaacggaaaagcaggattcctccgaggggaaaccctcgctggagcaatctccagcagtcctggacaagcctggtggtcagaagtcagccctgcctcaaccagcattcaagccccatccaaagtttttaaaggaatcccacgaagatgggaaagcaagagacaaagccatccaccaagccaacctgagacgtctctgccgcatctgtgggaattctttcaacaccactgggcacaagagaaggtatccagtccacgggcctgtggatggtaaaacccaagtccttttacggaagaaggaaaagagggccacgtcctggccagacctcattgccaaagttttccggatcgatgtgaaggcagatgttgactcgatccaccccactgagttctgccataactgctggagcttcatgcacaggaagtttagcagcaccccatgtgaggtttactccccaaggaatgcagccatggagtggcacccccacaccctaaactgtgacatctgccacattgcacgtcggggactcaagaggaagagtcagcagccaaacatgcagctcagcaaaaaactcaaaactgtgattgaccgagcgagacaagcccgtcagcgcaagaggagagctcaggccaggatcagcagcaaggaactgatgaagaagatcgccaactgcggtcagatacatcttagccccaagctcctggcagtggacttcccggcgcactttgtgaaatctatctcctgccagatttgtgaacacatcctggccgacccggtggagaccagctgcaagcatgtgttttgcaggatctgcattctcaggtgcctcaaagtcatgggcagcagttgtccctcttgccactatccctgttttcctactgacctggagagtccagtgaagtcttttctgagcatcttgaataccctgatggtgaaatgcccagcaaaggagtgcaacgaggagatcagcttggaaaaatataatcaccatatctcaagccacaaggagtcgaaggagacatttgtgcatattaataaagggggccggccccgccagcatctcctgtccctgacgcggagggctcagaaacaccgtctgagggagctcaagctgcaagtcaaggccttcgccgacaaagaagaaggtggcgatgtgaagtcagtgtgcctgaccttgttcctgctagtgctgagggcgaggaatgagcacagacaagctgacgagctggaggccatcatgcgaggccagggctccggcctgcagcctgctgtttgcttggccatccgcgtcaacaccttcctcagctgcagccagtaccacaagatgtacaggactgtgaaggccatcacgggcaggcagattttccagcctttgcatgcccttcggaatgcggagaaggtccttctgcccggctaccaccccttcgagtggcagccacctctgaagaatgtgtcttccagcacagacgtgggcattattgatgggctgtctggactctcctcctctgtggacgattacccagtggacaccattgccaagcgcttccgctatgactcggctctggtgtccgctctcatggacatggaagaagacatcctggagggtatgagagcccaagaccttgacgactacctgaatggccccttcactgtggtggtgaaggagtcttgtgatgggatgggagacgtgagtgagaagcacggcagtgggccggtcgtgccggaaaaggccgttcggttttccttcacagtcatgaaaatcaccatcgcacacgggtcacagaacgtgaaggtgtttgaggaagccaagcctaactctgaactatgctgcaagcccttgtgcctcatgctggccgacgaatccgaccatgagaccctgacggccatcctgagccctctcattgccgagagggaggccatgaagagcagccagctaatgctggagatgggaggcatcctccggactttcaagttcatcttcaggggcaccggatatgatgagaaactggtccgggaagtggaaggccttgaggcttctggctctgtctacatctgtaccctctgtgatgccacccgcctggaagcctctcaaaatctggtcttccactccataaccagaagccacgcggagaatttggagcgctatgaggtctggcgttccaacccataccatgagacggtggatgaacttcgggaccgggtgaaaggggtctcggccaaacccttcattgagacggtgccttccatagatgccctccactgtgacattggcaatgcagccgagttttacaagattttccagctcgagataggggaggcgtataagaacccccatgcctccaaggaggaaaggaagagatggcaggcgaccttggacaagcacctccgcaagaagatgaatctgaagcccatcatgagaatgaatggcaactttgccaggaagctcatgaccaaagagactgtggaagcagtctgtgagttaattccctccgaggagaggcatgaagctctgagggaactgatggacctttacctgaagatgaagcccgtctggcgatcatcatgccctgctaaagagtgcccggaatccctctgccagtatagtttcaattcacagcgttttgctgagctcctctccaccaagttcaagtacagatatgagggcaaaatcaccaattattttcacaagacactggcccacgtcccggaaattatcgagagggacggctccattggggcatgggctagcgagggaaatgagtctgggaacaagctgttcaggcgcttccgaaaaatgaatgccaggcagtccaagtactatgaaatggaagatgttttgaaacatcactggttgtacacctccaaatacctgcagaagtttatgaatgctcataaagcatttaaaaactcagggtttaccataaacttgcagagaagctcaggggacacattagacctagagaactctccagaatctcaagatttgatggaattttaagcagggaagttgcctaggcattggttttgcaaatgggttttctctgggttgcatggagagcttctcctggcacccttaactcctgggtgtggggcttcaccacccaagtggtggtaggttgatgaggccagagatggtgaacccatgtcgggaataggtaactggtgagccgattgcttgagctgtttagggagttcagaaaagcaataggagaaaccagttatttgaaagctccatagctgagaacaggggtaactgcaggggaccagaggtgaacaaagatgtgtggagaggattgagtgatgccaaagtcaaagccaaggttgtcaaaggacagccagtgaagccagaaaggaatttgacttgtggtttccattccccctgcccaagttattctagtttatactgagaggtgtttttcatatcacttttgtagaattttttattttataaatttcacatattttggcttattttacaagatttaaaaaaggttttcaagaattcccgctgtggcacaatgtggcatcttgggagtgctgggacacaggttccatccccagcctggcgtggtgggttaaggatcc

SEQ ID NO: 3 - protein, 1043 aa, Sus.scrofa
MAVSLPPTLGLSSAPDEIQHPHIKFSEWKFKLFRVRSFEKAPEKAQTEKQDSSEGKPSLEQSPAVLDKPGGQKSALPQPAFKPHPKFLKESHEDGKARDKAIHQANLRRLCRICGNSFNTTGHKRRYPVHGPVDGKTQVLLRKKEKRATSWPDLIAKVFRIDVKADVDSIHPTEFCHNCWSFMHRKFSSTPCEVYSPRNAAMEWHPHTLNCDICHIARRGLKRKSQQPNMQLSKKLKTVIDRARQARQRKRRAQARISSKELMKKIANCGQIHLSPKLLAVDFPAHFVKSISCQICEHILADPVETSCKHVFCRICILRCLKVMGSSCPSCHYPCFPTDLESPVKSFLSILNTLMVKCPAKECNEEISLEKYNHHISSHKESKETFVHINKGGRPRQHLLSLTRRAQKHRLRELKLQVKAFADKEEGGDVKSVCLTLFLLVLRARNEHRQADELEAIMRGQGSGLQPAVCLAIRVNTFLSCSQYHKMYRTVKAITGRQIFQPLHALRNAEKVLLPGYHPFEWQPPLKNVSSSTDVGIIDGLSGLSSSVDDYPVDTIAKRFRYDSALVSALMDMEEDILEGMRAQDLDDYLNGPFTVVVKESCDGMGDVSEKHGSGPVVPEKAVRFSFTVMKITIAHGSQNVKVFEEAKPNSELCCKPLCLMLADESDHETLTAILSPLIAEREAMKSSQLMLEMGGILRTFKFIFRGTGYDEKLVREVEGLEASGSVYICTLCDATRLEASQNLVFHSITRSHAENLERYEVWRSNPYHETVDELRDRVKGVSAKPFIETVPSIDALHCDIGNAAEFYKIFQLEIGEAYKNPHASKEERKRWQATLDKHLRKKMNLKPIMRMNGNFARKLMTKETVEAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKYYEMEDVLKHHWLYTSKYLQKFMNAHKAFKNSGFTINLQRSSGDTLDLENSPESQDLMEF

(19) United States
(12) Patent Application Publication
(10) Pub. No.: US 2005/0155094 A1
(43) Pub. Date: Jul. 14, 2005

(54) Swine rag-1 gene and utilization thereof

(76) Inventors: Hiroshi Yasue, Ibaraki (JP); Satoshi Watanabe, Ibaraki (JP); Daisuke Honma, Ibaraki (JP); Yohtaroh Takagaki, Kanagawa (JP); Hiromi Hatsuse, Kanagawa (JP)

Correspondence Address: STERNE, KESSLER, GOLDSTEIN & FOX PLLC, 1100 NEW YORK AVENUE, N.W., WASHINGTON, DC, 20005, US

(21) Appl. No.: 10/503,464

(22) PCT Filed: Feb. 4, 2003
(86) PCT No.: PCT/JP03/01114

(30) Foreign Application Priority Data
Feb. 4, 2002 (JP) 2002-27450

(51) Int. Cl. A01K 67/027; C07K 14/705; C07K 16/28

(52) U.S. Cl. 800/17; 530/350; 530/388.22; 435/325; 435/320.1

(57) ABSTRACT

BAC clones comprising the porcine RAG-1 gene were obtained by screening a porcine genomic DNA library. Three clones were obtained by subcloning the region comprising the RAG-1 gene from the BAC clones. Genetic maps were obtained by determining the nucleotide sequence of these clones using sequencing, and by mapping using restriction enzyme digests. Based on this information, targeting vectors that can knockout the RAG-1 gene were constructed. Using these vectors it is possible to construct swine that do not possess acquired immunity by suppressing endogenous porcine RAG-1 gene function.

### TECHNICAL FIELD

The present invention relates to porcine RAG-1 genes and uses-thereof.

### BACKGROUND ART

In vertebrates, the acquired immune system is the major force against foreign pathogens. The acquired immune system is an antigen-specific immune condition acquired by the body's immune system as a result of stimulation by foreign substances (antigens) after birth. The acquired immune system is triggered by antigen recognition by T cell antigen receptors, or immunoglobulins produced by B cells. In the process of T cell and B cell differentiation, the genes in an embryonic cell genome are reshuffled and become activated genes, and T cell antigen receptors and immunoglobulins exert acquired immune activity. Dr. Susumu Tonegawa received the 1987 Nobel Prize in Physiology and Medicine for the discovery of gene recombination in the development of the acquired immune system.

Gene recombination is initiated by the cleavage of the Recombination Signal Sequence in genomic genes by the RAG-1 and RAG-2 protein complex (RAG-1 protein is the type 1 product of the Recombination Activation Gene). It is known that in the absence of the RAG-1 gene, T cell antigen receptors and immunoglobulins cannot be produced, resulting in severe combined immunodeficiency diseases, without acquired immunity. In fact, it is known that RAG-1 gene-deficient mice, obtained by knockout methods, cannot produce T cell antigen receptors and immunoglobulins (Mombaerts, P. et al., Cell, (1992) 68 (5), 869-77).

Such mice, which do not possess acquired immunity, can conceivably be used for xenotransplants, retrogenerative medicine, and such. However, there are problems in the application of these mice to such fields, since mouse organs are small compared with human organs, and mice are physiologically very different from humans (for example, the life expectancy of a mouse is about two years, and mice cells age more rapidly than human cells).

### DISCLOSURE OF THE INVENTION

The present inventors considered that swine, whose body fluid composition and physiological functions are similar to those of humans, and whose generation time is shorter than that of primates, were suitably sized animal for application in xenotransplants, retrogenerative medicine, and such. At this time there had been no reports of RAG-1 gene knockout swine. Identifying the porcine RAG-1 gene was prerequisite to constructing a knockout swine, however, there were also no reports of the isolation of a DNA coding for the porcine RAG-1 protein.

The present invention is based on the considerations described above. The first objective of the present invention is to provide DNA coding for the porcine RAG-1 protein. The present invention also aims to provide a vector expressing this DNA, transformants in which the vector has been introduced, porcine RAG-1 protein and methods for its production, and antibodies which bind to this protein.

Furthermore, an objective of the present invention is to construct swine in which the function of the porcine RAG-1 gene is suppressed. Another objective of the present invention is to provide vectors and cells for use in constructing these swine.

The present inventors obtained BAC clones comprising the porcine RAG-1 gene by screening a porcine genomic DNA library. Regions comprising the RAG-1 gene were subcloned from the BAC clones, and three clones were obtained. Genetic maps of these clones were constructed by determining their nucleotide sequences using sequencing, and by mapping using restriction enzyme digestion. Based on this information, targeting vectors for use in knocking-out the RAG-1 gene were constructed.

Specifically, the region from the KpnI recognition site just before the 5′ initiation codon of the RAG-1 gene, to the HincII recognition site approximately 1.4 Kb downstream of this KpnI site, was replaced with a puromycin resistance gene. The puromycin resistance gene is used for positive selection during gene transfer. This substitution prevents the RAG-1 gene from being expressed. The region for homologous recombination was the region from the XhoI recognition site approximately 9.0 Kb upstream of the initiation codon, to the XhoI site approximately 2.5 Kb downstream of the initiation codon. The DT-A gene was also inserted for negative selection during gene transfer. Swine without an acquired immune system can be constructed by suppressing the function of the endogenous RAG-1 gene using the constructed targeting vectors.

Therefore, the present invention relates to DNA coding for swine-derived RAG-1 protein and uses thereof, especially use for the construction of swine without an acquired immune system. More specifically, it provides:
(1) a porcine-derived DNA of any one of the following (a) to (c):
   (a) a DNA comprising the nucleotide sequence of SEQ ID NO: 1 or 2;
   (b) a DNA encoding a protein comprising the amino acid sequence of SEQ ID NO: 3;
   (c) a DNA encoding a protein comprising an amino acid sequence in which one or more amino acids in the amino acid sequence of SEQ ID NO: 3 have been added, deleted, substituted, and/or inserted, and also comprising a DNA recombination inducing activity;
(2) a vector comprising the DNA of (1);
(3) a transformed cell containing the DNA of (1), or the vector of (2);
(4) a protein encoded by the DNA of (1);
(5) a method for producing the protein of (4), comprising a step of culturing the transformed cell of (3), and recovering an expressed protein from the media of the culture;
(6) an antibody which binds to the protein of (4);
(7) a vector comprising a DNA sequence which suppresses the function of endogenous porcine RAG-1 gene by homologous recombination;
(8) a porcine cell into which the vector of (7) is introduced;
(9) a method for producing porcine in which endogenous RAG-1 gene function is suppressed, comprising the following steps (a) and (b):
   (a) implanting a nucleus of the cell of (8) into an enucleated egg;
   (b) implanting the enucleated egg obtained in (a) into a swine;
(10) a method for producing a swine with suppressed endogenous RAG-1 gene function, comprising a step of implanting the cell of (8) into the swine.

The present invention provides DNAs encoding porcine RAG-1 protein. In the present invention, “RAG-1 protein” indicates a type 1 product of the RAG protein complex necessary for initiation of gene recombination. In the present invention, RAG-1 protein activity can be measured as the activity of inducing DNA recombination. The activity of inducing DNA recombination can be measured using well known methods (Agawal. A., Eastmanq. M. and Schatz D. G. (1998) Nature, 394. 744-751). The RAG-1 gene sequence and RAG-1 cDNA sequence, which were revealed by the present invention, are indicated as SEQ ID NO: 1 and SEQ ID NO: 2, respectively, and the amino acid sequence of the RAG-1 protein encoded by these DNAs is indicated in SEQ ID NO: 3.

The present invention also includes DNAs coding for variants of the porcine RAG-1 protein. Methods well known to those skilled in the art are used to prepare these proteins, and include known methods of inducing mutations in proteins. For example, one skilled in the art can prepare proteins functionally equivalent to porcine RAG-1 protein (the protein comprising the amino acid sequence described by SEQ ID NO: 3) by inducing appropriate mutations in to its amino acid sequence using site-directed mutagenesis (Hashimoto-Gotoh, T. et al., Gene (1995) 152, 271-275; Zoller, M J, and Smith, M., Methods Enzymol. (1983) 100, 468-500; Kramer, W. et al., Nucleic Acids Res. (1984) 12, 9441-9456; Kramer W, and Fritz H J., Methods. Enzymol. (1987) 154, 350-367; Kunkel, T A, Proc. Natl. Acad. Sci. USA. (1985) 82, 488-492; Kunkel, Methods Enzymol. (1988) 85, 2763-2766). Amino acid mutations can also occur in nature. The proteins of the present invention also include proteins comprising the amino acid sequence of porcine RAG-1 protein in which one or more amino acids are mutated, provided that the resulting mutated proteins are functionally equivalent to porcine RAG-1 protein. The number of amino acids to be mutated in such mutants is generally 50 amino acids or less, preferably 30 amino acids or less, and more preferably ten amino acids or less (for example, five amino acids or less).

A mutated amino acid residue is preferably mutated to a different amino acid that conserves the properties of the amino acid side-chain. Examples of the properties of amino acid side chains include hydrophobic amino acids (A, I, L, M, F, P, W, Y, V), hydrophilic amino acids (R, D, N, C, E, Q, G, H, K, S, T) and amino acids comprising an aliphatic side-chain (G, A, V, L, I, P), a hydroxyl group-containing side-chain (S, T, Y), a sulfur atom containing side-chain (C, M), a carboxylic acid- and amide-containing side-chain (D, N, E, Q), a base-containing side-chain (R, K, H), or an aromatic-containing side-chain (H, F, Y, W) (The parenthetic letters indicate the one-letter amino acid codes).

Proteins comprising amino acid sequences that are modified from a given amino acid sequence, by the deletion, addition, and/or substitution with another amino acid, of one or more amino acid residues, are already known to retain their biological activity (Mark, D. F. et al., Proc. Natl. Acad. Sci. USA (1984) 81, 5662-5666, Zoller, M. J. & Smith, M., Nucleic Acids Research (1982) 10, 6487-6500, Wang, A. et al., Science 224, 1431-1433, Dalbadie-McFarland, G. et al., Proc. Natl. Acad. Sci. USA (1982) 79, 6409-6413).

Fusion proteins are included as proteins to which more than one amino acid residue has been added to the porcine RAG-1 protein amino acid sequence. Fusion proteins are fusions of porcine RAG-1 protein and other peptides or proteins, and are included in the present invention. Fusion proteins can be made by techniques well known to those skilled in the art, such as by linking the DNA that encodes porcine RAG-1 protein (comprising the amino acid sequence described in SEQ ID NO: 3) with a DNA encoding another peptide or protein so that their frames match, inserting this fused DNA into an expression vector, and expressing it in a host. There are no restrictions as to the peptides or proteins fused to the protein of the present invention.

Examples of known peptides that can be used as the other peptide, which is fused to porcine RAG-1 protein, include FLAG (Hopp, T. P. et al., Biotechnology (1988) 6, 1204-1210), 6×His containing six His (histidine) residues, 10×His, HA (Influenza agglutinin), human c-myc fragments, VSP-GP fragments, p18HIV fragments, T7-tag, HSV-tag, E-tag, SV40T antigen fragments, lck tag, α-tubulin fragments, B-tag, Protein C fragments, and the like. Examples of proteins that may be fused to porcine RAG-1 protein include GST (glutathione-S-transferase), HA (Influenza agglutinin), immunoglobulin constant regions, β-galactosidase, MBP (maltose-binding protein), and such. Fusion proteins can be prepared by fusing commercially available DNAs, which encode the above fusion peptides or proteins, with a DNA encoding the protein of the present invention, and expressing the fused DNA thus prepared.

The present invention also provides vectors comprising the above-mentioned DNAs, transformants in which the vectors have been introduced, proteins coded by the above-mentioned DNAs and production method thereof, and antibodies that bind to these proteins.

Vectors generally used by those skilled in the art can be applied to the present invention. For example, if E. coli is used as a host for transformation, it is preferable to use, for example, vectors comprising “ori”, which is required for replication in E. coli, and genes necessary for the selection of transformed E. coli (for example, genes resistant to drugs (such as Ampicillin, Tetracycline, Kanamycin, and Chloramphenicol)) These vectors include, for example, M13 type vector, pUC type vector, pBR322, pBluescript, pCR-Script, pGEM-T, pDIRECT, and pT7.

An expression vector is especially useful when a vector is used to produce RAG-1 protein. For example, if aiming for expression in E. coli, expression vectors include vectors comprising a lacZ promoter (Ward et al., Nature (1989) 341, 544-546; FASEB J (1992) 6, 2422-2427), araB promoter (Better et al., Science (1988) 240, 1041-1043), T7 promoter or such. In addition to the above vectors, examples of these kinds of vectors include pGEX-5X-1 (Pharmacia), “QIAexpress system” (Qiagen), pEGFP, and pET (in this case, the host is preferably T7 RNA polymerase-expressing BL21). Additionally, the expression vectors may also contain signal sequences for polypeptide secretion. The pelB signal sequence or the like can be used as the signal sequence for protein secretion when, for example, the protein is to be produced in the periplasm of E. coli (Lei, S. P. et al J. Bacteriol. (1987) 169, 4379).

Other vectors for producing RAG-1 protein include, for example, expression vectors derived from mammals (for example, pcDNA3 (Invitrogen) and pEGF-BOS (Nucleic Acids. Res. (1990) 18 (17), p5322), pEF, pCDM8), expression vectors derived from insect cells (for example, “Bac-to-BAC baculovirus expression system” (GIBCO BRL), pBacPAK8), expression vectors derived from plants (for example pMH1, pMH2), expression vectors derived from animal viruses (for example, pHSV, pMV, pAdexLcw), expression vectors derived from retroviruses (for example, pZIpneo), expression vector derived from yeast (for example, “Pichia Expression Kit” (Invitrogen), pNV11, SP-Q01), and expression vectors derived from Bacillus subtilis (for example, pPL608, pKTH50).

When aiming to express a vector in animal cells, such as CHO, COS, or NIH3T3 cells, the vector should comprise a promoter necessary for expression in such cells, for example, SV40 promoter (Mulligan et al., Nature (1979) 277, 108), MMLV-LTR promoter, the EF1α promoter (Mizushima et al., Nucleic Acids Res. (1990) 18, 5322), CMV promoter, or the like, and preferably comprise a marker gene for selecting transformed cells (for example, genes that are resistant to drugs (e.g., neomycin, G418 and such)). Examples of known vectors with these characteristics include, for example, pMAM, pDR2, pBK-RSV, pBK-CMV, pOPRSV, and pOP13.

A method when aiming to express a gene stably and to amplify the gene copy number in cells is, for example, the introduction of a vector comprising a complementary DHFR gene (for example pCHO I) into CHO cells with a defective pathway for nucleic acid synthesis, and then amplifying using methotrexate (MTX). Furthermore, when aiming for transient expression of a gene, a method can be used wherein a vector comprising a SV40 replication origin (pcD, etc.) is transfected into COS cells comprising the SV40 T antigen-expressing gene in their chromosomes.

Moreover, the origin of replication can be derived from a polyomavirus, adenovirus, bovine papilomavirus (BPV), and such. Furthermore, expression vectors can contain aminoglycosid transferase (APH) gene, thymidine kinase (TK) gene, E. coli xanthine guanine phosphoribosyl transferase (Ecogpt) gene, dihydrofolic acid reductase (dhfr), and such as selective markers for amplification of gene copy number in a host cell system.

The host cell into which the vector is transfected is not particularly limited. For example, E. coli and various types of eukaryote cells and such can be used. For example, when using eukaryotic cells, animal, plant, or fungi cells can be used as a host. Exemplary animal cells include, for example, mammalian cells such as CHO, COS, 3T3, myeloma, baby hamster kidney (BHK), HeLa, or Vero cells, amphibian cells such as Xenopus oocytes (Valle et al. Nature (1981) 291: 340-358), or insect cells such as Sf9, Sf21, or Tn5 cells. Particularly suitable CHO cells are DHFR-gene-deficient CHO cells dhfr-CHO (Proc. Natl. Acad. Sci. U.S.A. (1980) 77: 4216-4220) or CHO K-1 (Proc. Natl. Acad. Sci. U.S.A. (1968) 60: 1275). Of the animal cells, CHO cells are particularly preferable when aiming for abundant expression. Nicotiana tabacum-derived cells are examples of the plant cells. Known fungi cells include yeast cells such as Saccharomyces, including Saccharomyces cerevisiae, or filamentous fungi such as Aspergillus, including Aspergillus niger. When using prokaryotic cells, examples of the bacterial cells include E. coli, for example, JM109, DH5a, HB101, XLlBlue and BL21. In addition, Bacillus subtilis is also known.

When using animals as hosts, mammals, plants, and insects can be used. As the mammals, goats, swine, sheep, mice, and bovine can be used (Vicki Glaser, SPECTRUM Biotechnology Applications (1993)). Furthermore, for example, tobacco can be used when using plants. Silkworms can be used as the insects, for example.

In order to construct the transformants of the present invention, vectors are introduced into the above-described hosts. Methods for introducing vectors into host cells such as E. coli include, for example, calcium chloride methods and electroporation methods (Chu, G. et al., Nucl. Acid. Res. (1987) 15, 1311-1326). When the host cells are cultured cells, vectors can be introduced by, for example, calcium phosphate methods (Chen, C. and Okayama, H. Mol. Cell. Biol. (1987) 7, 2745-2752), DEAE Dextran methods (Lopata, M. A. et al., Nucl. Acid. Res. (1984) 12, 5707-5717, Sussman, D. J. and Milman, G. Mol. Cell. Biol. (1985) 4, 1642-1643), cationic liposome DOTAP methods (Boehringer Mannheim), and lipofectin methods (Derijard, B. Cell (1994) 7, 1025-1037, Lamb, B. T. et al., Nature Genetics (1993) 5, 22-30, Rabindran, S. K. et al., Science (1993) 259, 230-234).

Furthermore, when introducing DNAs into animals, each DNA can be inserted into an appropriate vector (for example, adenovirus vectors (for example pAdexlcw) and retrovirus vectors (for example pZIPneo), without limitation), and introduced into living bodies using methods such as retrovirus methods, liposome methods, cationic liposome methods, and adenovirus. Moreover, vectors can be introduced into insects by, for example, infecting silkworms with a baculovirus comprising a DNA that encodes a target protein (Susumu, M. et al., Nature (1985) 315, 592-594). Moreover, when the DNA is to be introduced into plants, for example, DNAs encoding target proteins can be inserted into plant expression vectors such as pMON 530, and introduced into bacteria such as Agrobacterium tumefaciens. The vectors can be introduced by infecting these bacteria into tobacco, for example, Nicotiana tabacum (Julian K.-C. Ma et al., Eur. J. Immunol. (1994) 24, 131-138).

The RAG-1 proteins of the present invention can be produced, for example, by culturing transformants of the present invention. Cultures can be carried out according to known methods. For example, culture media that can generally be used when culturing animal cells include DMEM, MEM, RPMI1640, or IMDM. These media may be used with or without serum supplements such as fetal calf serum (FCS). The pH of the culture medium is preferably between about 6 and 8. Such cells are typically cultured at about 30° C. to 40° C. for about 15 to 200 hours, and culture media may be replaced, aerated, or stirred as necessary.

The RAG-1 proteins of the present invention may be isolated from inside or outside (such as from the medium) of host cells, and can be purified as substantially pure homogeneous proteins. General methods for protein isolation and purification can be used without limitation. For instance, column chromatography, filtration, ultrafiltration, salt precipitation, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the protein. Alternatively, protein purification can also be performed by using these columns in multiple combinations.

Examples of chromatography include, for example, affinity chromatography in which antibodies against RAG-1 proteins of the present invention are immobilized, ion-exchange chromatography, hydrophobic chromatography, gel filtration, reverse phase chromatography, adsorption chromatography, and such (Strategies for Protein Purification and Characterization: A Laboratory Course Manual. Ed. Daniel R. Marshak et al., Cold Spring Harbor Laboratory Press (1996)). These chromatographies may be performed by liquid chromatography, such as HPLC and FPLC. By use of these purification methods, RAG-1 protein of the present invention can be highly purified.

A protein of the present invention may be optionally modified or partially deleted by treatment with an appropriate protein modification enzyme before or after purification. Useful protein modification enzymes include, but are not limited to, trypsin, chymotrypsin, lysylendopeptidase, protein kinase, glucosidase, and so on.

When RAG-1 proteins of the present invention are expressed in host cells (for example, animal cells and E. coli) as fusion proteins with Glutathione S-transferase protein, or as recombinant proteins in which multiple histidine residues are attached, the expressed recombinant proteins can be purified using a glutathione column or a nickel column. After purifying the fusion proteins, regions other than the target protein can be cleaved with thrombin, factor Xa, or such, and removed as necessary.

Furthermore, proteins can also be recovered from systems in which individuals produce a protein. Systems in which proteins are produced by individual bodies include, for example, production systems using animals and plants. Proteins can be recovered after introducing target DNAs into animals or plants, and producing the proteins in the animal or plant bodies.

For instance, a desired DNA may be prepared as a fusion gene, by fusing it with a gene that encodes a protein specifically produced in milk, such as goat β casein gene. DNA fragments comprising the fusion gene are injected into goat embryos, which are then impregnated into female goats. Target proteins can be recovered from milk produced by the transgenic goats (i.e., born from goats that received the modified embryos) or from their offspring. To increase the amount of protein-comprising milk produced by the transgenic goats, appropriate hormones may also be applied to these goats (Ebert K. M. et al. Bio/Technology (1994) 12: 699-702).

When using insects, target proteins can also be recovered from, for example, the body fluids of silkworms infected with a Baculovirus comprising a DNA encoding a target protein, (Susumu, M et al., Nature (1985) 315, 592-594).

Furthermore, when using plants, desired proteins can be recovered from, for example, tobacco (tobacco leaves) regenerated using methods well known to those skilled in the art (Toki et al., Plant Physiol (1995) 100, 1503-1507) from Nicotiana tabacum-derived cells in which a vector has been introduced by an above-mentioned method (Julian K.-C. Ma et al., Eur. J. Immunol. (1994) 24, 131-138).

Antibodies which bind to the RAG-1 proteins of the present invention can be used for purification and detection of RAG-1 protein. Antibodies used for the purification and detection of RAG-1 protein in the present invention can be produced by known methods. There is no particular limit to the form of the antibodies, which include antiserum obtained by immunizing immune animals such as rabbits with antigenic protein, all classes of polyclonal and monoclonal antibodies, as well as human antibodies and humanized antibodies produced by genetic recombination. Furthermore, antibodies can be antibody fragments and modified antibodies as long as they bind to RAG-1 protein.

Antibodies can be obtained by using RAG-1 protein as a sensitizing antigen. RAG-1 proteins for use as sensitizing antigens can be either full-sized proteins or partial peptides of proteins. Moreover, protein-expressing cells or their lysates, or chemically synthesized RAG-1 proteins can be used as sensitizing antigens. Short peptides can be used as antigens by properly binding to carrier proteins such as keyhole limpet hemocyanin, bovine serum albumin, or Ovalbumin.

The mammals immunized by sensitizing antigens are not limited, however, they are preferably selected after consideration of their suitability with the parent cells used for cell fusion in monoclonal antibody production. Animals generally used include rodents such as mice, lagomorphs such as rabbits, or primates such as rhesus monkeys. Methods for immunizing animals with sensitizing antigens are well known to those skilled in the art.

Polyclonal antibodies, for example, may be obtained by collecting blood from antigen-sensitized mammals in which an increase in desired antibodies in the serum has been confirmed, and separating serum from this blood using conventional methods. Serum comprising polyclonal antibodies can be used as the polyclonal antibodies, and fractions comprising polyclonal antibodies may be further isolated from this serum as necessary.

To prepare monoclonal antibodies, for example, an increase in desired antibodies is confirmed in the serum of the above-mentioned antigen-sensitized mammals, and then immune cells are collected and subjected to cell fusion. In this case, spleen cells are specific examples of immune cells preferably used for cell fusion. Other parent cells to be fused with the above immune cells preferably include, for example, mammalian myeloma cells, and more preferably myeloma cells that have acquired a property for selecting fused cells using drugs. The above immune cells and myeloma cells can be fused according to known methods, for example, the method of Milstein et al. (Galfre, G. and Milstein, C., Methods Enzymol. (1981) 73, 3-46).

Next, using known methods, hybridomas that produce the desired antibodies are cloned from the hybridomas obtained by cell fusion. The cloned hybridomas are implanted into mouse peritoneal cavities, and then ascitic fluid is recovered from the mice.

Obtained antibodies may be purified to homogeneity. Antibody separation and purification can be performed according to separation and purification methods used for general proteins. For example, the antibodies may be separated and isolated by the appropriate selection and combined use of column chromatography such as affinity chromatography, filtration, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but these methods are not limiting. Protein A and protein G columns can be used as columns for affinity chromatography. Exemplary protein A columns include, for example, Hyper D, POROS, and Sepharose F.F. (Pharmacia).

For example, the antigen-binding activities of the antibodies can be measured by methods such as absorbance measurement, enzyme-linked immunosorbent assays (ELISA), enzyme immunoassays (EIA), radioimmunoassays (RIA), and/or immunofluorescence. In ELISA, the antibodies are immobilized on a plate, RAG-1 protein is added to the plate, and then a sample comprising a desired antibody, for example the culture supernatant of antibody-producing cells or purified antibodies, is added. A secondary antibody, which recognizes the primary antibody and is labeled with an enzyme such as alkaline phosphatase, is then added, and the plate is incubated. Next, after washing, an enzyme substrate such as p-nitrophenyl phosphate is added to the plate, and absorbance is measured to evaluate the sample's antigen-binding activity. Fragments of RAG-1 protein can also be used as a protein that is added to the plates.

The present invention also provides vectors (targeting vectors) that comprise DNA sequences which suppress endogenous porcine RAG-1 gene function by homologous recombination. Individual swine in which endogenous RAG-1 gene function is suppressed (the RAG-1 gene is knocked out) can be constructed using these vectors. Suppression of RAG-1 gene function by such vectors can be performed by suppressing the expression of the RAG-1 gene, by inducing a mutation in the expression control region of the RAG-1 gene. Furthermore, this suppression can be performed by inducing a mutation in the exon region of the RAG-1 gene to express a mutant RAG-1 protein, whose activity is suppressed compared with the activity of wild type RAG-1 protein. “Suppression” of RAG-1 gene function includes both complete and partial suppression of the RAG-1 gene.

The vectors that comprise a DNA sequence which suppresses porcine endogenous RAG-1 gene function by homologous recombination are not limited, as long as they are able to induce mutations which cause suppression of endogenous porcine RAG-1 gene function. These vectors include, for example, vectors which comprise a mutated expression control region or exon region of the porcine RAG-1 gene, and/or a homologous recombination region. Moreover, vectors additionally comprising a gene for positive selection (selection to choose only cells in which DNA is introduced) or for negative selection (selection to exclude non-homologous recombinants) are also included. Furthermore, vectors which also comprise a sequence coding for an enzyme which enhances recombination in the RAG-1 gene site, for example, Cre for Cre-lox, are also included.

Induction of mutations can be performed, for example, by using a gene for positive selection as described in the Examples, however, there is no limit to the induction of mutations as long as endogenous RAG-1 gene function can be suppressed. There is no specific limitation as to the genes used for positive selection, and examples include puromycin resistance gene and neomycin resistance gene. Genes used for negative selection include thymidine kinase gene and diphtheria toxin gene.

In the present invention, a homologous recombination region indicates a DNA sequence which mediates homologous recombination when a mutated expression control region or exon region of a porcine RAG-1 gene is introduced into genomic DNA. Therefore, there is no limit to the homologous recombination region in the present invention, as long as the DNA sequence can mediate homologous recombination. Those skilled in the art can ordinarily carry out selection of DNA sequences.

Moreover, the present invention provides porcine cells in which targeting vectors of the present invention are introduced. The porcine cells include somatic cells, fertilized eggs, and such, but are not limited thereto. In the present invention, methods known to those skilled in the art can be used to introduce targeting vectors into the above-mentioned cells. Specifically, electroporation methods can be performed. For example, 1×10⁷ cells and 50 nM targeting vector are suspended in 500 μl HEPES buffer and transferred into a 4.0 mm gapped electroporation cuvette. A gene is introduced into the cell by applying one pulse using an electroporator at a setting of 250V, 960 μF, and infinite resistance (values can vary depending on cell origin).

Moreover, the present invention provides methods to produce RAG-1 gene knockout swine (endogenous RAG-1 gene function is suppressed). These methods are not particularly limited, and for example include methods where an enucleated egg is implanted with a nucleus from a somatic cell in which endogenous RAG-1 gene function is suppressed, and the obtained enucleated egg is then implanted into swine, which can give birth to individual swine. Other methods include methods of directly injecting a targeting vector of the present invention into fertilized eggs, and then implanting the fertilized eggs into swine, which can give birth to individual swine.

### BRIEF DESCRIPTION OF THE DRAWINGS

FIG. 1 is a schematic illustration of a vector in which the RAG-1 gene has been subcloned. K, X, S, H, B, and N indicate KpnI, XhoI, SacI, HincII, BamHI, and NotI, respectively. (a) indicates genomic DNA, (b) indicates RAG-1-BAC-SacI, (c) indicates Rag1-Clawn-genome-BamHI, (d) indicates pBKS-RagKp.

FIG. 2 is a schematic illustration of a genetic map of the RAG-1 gene region. The diagonal box (1) indicates the first exon, diagonal box (2) indicates the second exon, and the region between (1) and (2) indicates an intron. The thick line (3) indicates the region used for the newly devised probe (e.g. RAG-1 Koitabashi probe) for cloning. The thick line (4) indicates the region used as a probe for subcloning from BAC (intron 5′ upstream probe). The region between (A) and (B) indicates the Clawn-genome-BamHI subcloning site, and the region between (C) and (D) indicates the BAC-SacI subcloning site.

FIG. 3 is a schematic illustration of the targeting vector and a genetic map of the RAG-1 gene region before and after RAG-1 targeting. K, X, S, H, B, Sal, and N indicate Kpn, XhoI, SacI™ HincII, BamHI, SalI, and NotI, respectively. (a) indicates genomic DNA, (b) indicates a targeting vector, (c) indicates a target genome.

FIG. 4 is a schematic illustration of the process of constructing the RAG-1 gene targeting vectors. K, X, S, H, B, Sal, and N indicate KpnI, XhoI, SacI, HincII, BamHI, SalI, and NotI respectively.

### BEST MODE FOR CARRYING OUT THE INVENTION

The present invention is specifically illustrated below with reference to Examples, but it is not to be construed as being limited thereto.

### EXAMPLE 1

### Construction of a Targeting Vector for Porcine RAG-1 Gene Knockout

First, the isolation of porcine RAG-1 cDNA was attempted. At this time, known cDNA could not be obtained. Furthermore, after detailed investigation of information on known mouse and human nucleotide sequences, it was determined that porcine RAG-1 cDNA might not cross-hybridize with probes derived from other species. Therefore, reverse transcription PCR from porcine mRNA was performed. First, four PCR primers known for their use for the human RAG-1 gene were used (Chun et al., Cell (1991) 64: 189-200); however, PCR product from swine was not obtained. Therefore, as the result of the trial and error production of a large number of primers, using mice and human nucleotide sequence consensus regions as leads, a 1161 bp cDNA fragment (the region from 7010 bp to 8171 bp in the attached 9.1 Kb nucleotide sequence) with 88% homology to the human RAG-1 cDNA nucleotide sequence was cloned. A porcine thymus cDNA library was screened using this cDNA fragment as a probe, and a cDNA clone covering about 80% of the full-length was obtained. The rest of the cDNA nucleotide sequence was obtained using 5′ and 3′ RACE methods. Thus, cloning porcine RAG-1 cDNA in sections was extremely difficult.

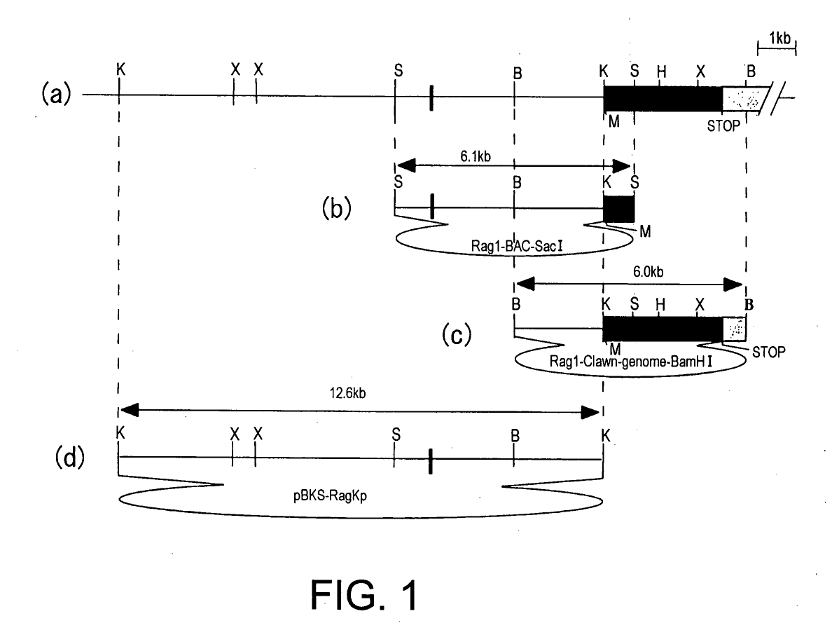

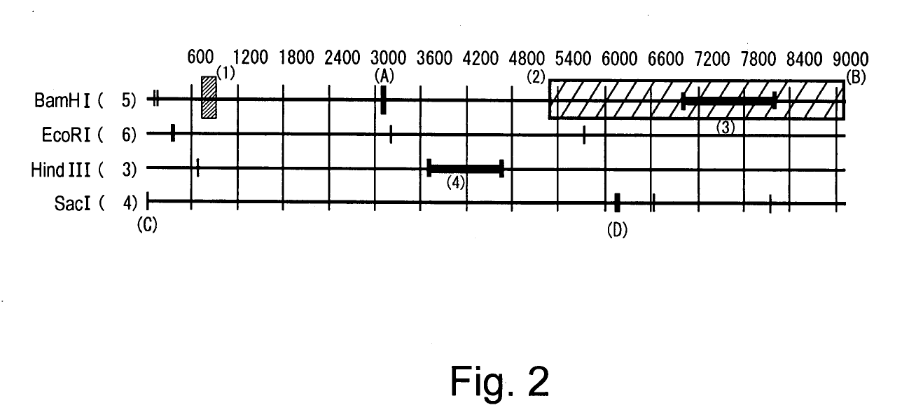

Next, PCR primers were constructed based on the information obtained from this cDNA. A porcine genomic DNA library was screened using the constructed PCR primers, and BAC clone 578B12, which comprises the porcine RAG-1 gene, was obtained. The region of the BAC clone that comprised the RAG-1 gene was subcloned, and three clones (approximately 6.1 Kb Rag1-BAC-SacI, approximately 6.0 Kb Rag1-Clawn-genome-BamHI, and approximately 12.6 Kb pBKS-RagKp) were obtained (FIG. 1). The nucleotide sequences of these clones were determined by sequencing (approximately 9.1 Kb) (SEQ ID NO: 1), and were mapped by restriction enzyme digests in order to construct an approximately 16 Kb genetic map (FIGS. 1 and 2). The targeting vectors for knockout of the RAG-1 gene were constructed based on this information.

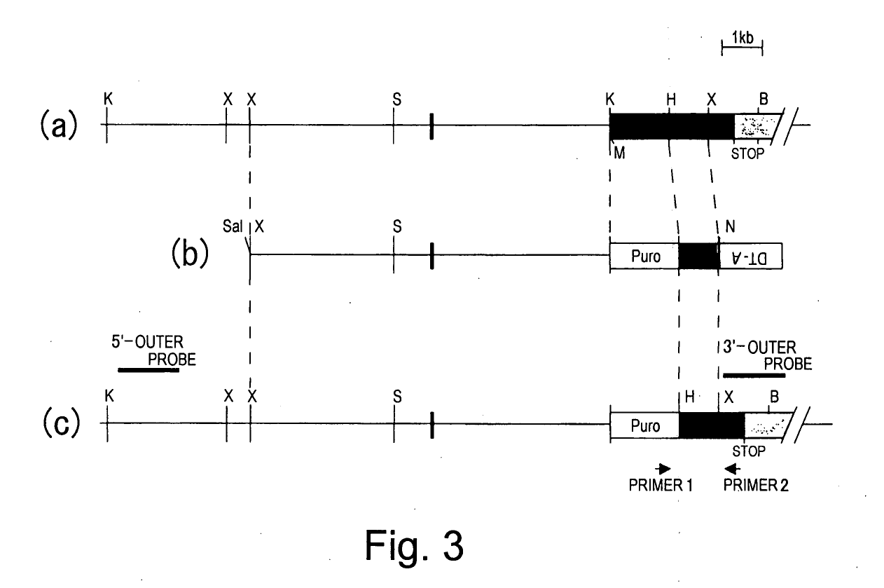

Using this targeting vector, the region from the KpnI recognition site, located immediately 5′ of the RAG-1 gene initiation codon, to the HincII recognition site, located approximately 1.4 Kb downstream from the KpnI site, was replaced with a puromycin resistance gene (FIG. 3). This puromycin resistance gene is utilized for positive selection during gene transfer. The RAG-1 gene cannot be expressed because of this substitution. The region for homologous recombination is defined as the region between the XhoI recognition site, located approximately 9.0 Kb upstream of the initiation codon, and the XhoI recognition site, located approximately 2.5 Kb downstream of this initiation codon. Furthermore, the DT-A gene is linked for use for negative selection during gene transfer (FIG. 3).

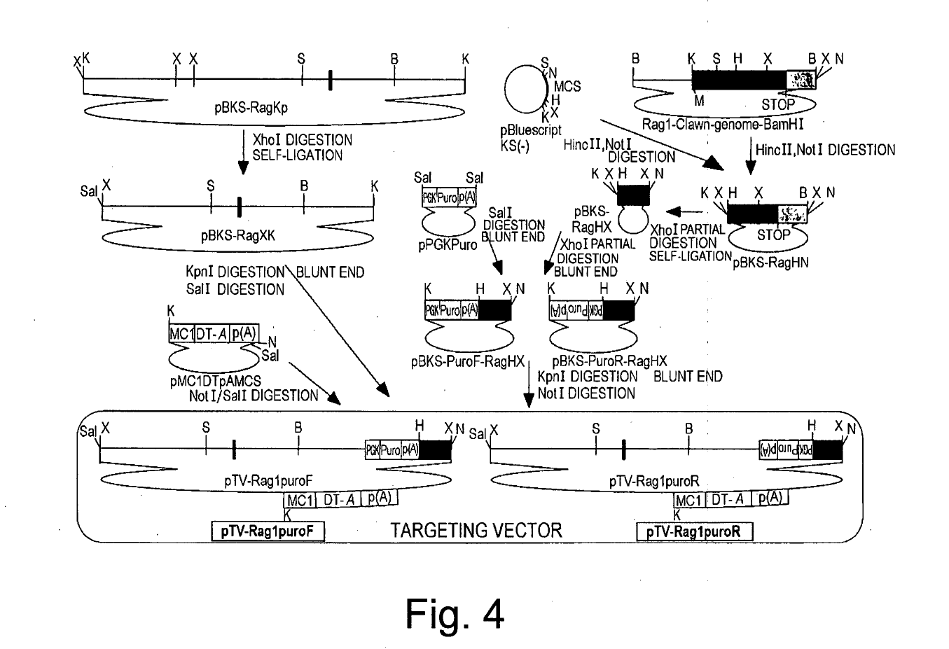

First, pBKS-RagKp, which comprised the 5′ side of the homologous recombination region, was digested with XhoI and self-ligated to obtain pBKS-RagXK. Furthermore, Rag1-Clawn-genome-BamHI, which comprised the 3′ side of the homologous recombination region, was digested with HincII and NotI to obtain an approximately 2.2 Kb fragment. pBKS-RagHN was obtained by ligating this fragment to pBluescriptKS(−), which had been digested with HincII and NotI in the same way. This vector was then partially digested with XhoI and self-ligated to obtain pBKS-RagHX, in which the approximately 1.2 Kb comprising the stop codon was removed. The approximately 4.0 Kb fragment obtained by partially digesting pBKS-RagHX with XhoI, was ligated to the approximately 1.7 Kb fragment obtained by digesting pPGK-puro, which comprised the puromycin resistance gene, with SalI. Thus, two different types of vectors with opposite orientation of the puromycin resistance gene were obtained: pBKS-PuroF-RagHX and pBKS-PuroR-RagHX. These two vectors were digested with KpnI and NotI, and the approximately 2.7 Kb fragment thus obtained was linked with the approximately 9.0 Kb fragment obtained by digesting the above-mentioned pBKS-RagXK with SalI and KpnI, and an approximately 4.3 Kb fragment obtained by digesting a pMClDTpAMCS vector comprising the DT-A gene with NotI and SalI. The targeting vectors pTV-Rag1puroF and pTV-Rag1puroR were thus obtained (FIG. 4).

### Industrial Applicability

Swine without T cell antigen receptors and immunoglobulins can be obtained using the targeting vectors of the present invention. This results in swine that lack acquired immunity, rendering several important applications possible.

(1) Miniature Swine for Xenotransplants

At the present time, due to advances in technology, the demand for organs for organ transplant from brain-dead individuals is more than five times that of supply. Therefore, there is a serious undersupply of organs, such that there is even an organ black market. Modified miniature swine that carry human genes for organ transplant into humans have already been developed by several European and American venture companies, and the transplanted condition has been successfully maintained for several months in primates such as baboons. In these miniature swine for use in xenotransplants, rejection that arises at a relatively early stage, such as hyperacute vascular rejection, does not occur. However, in order to maintain transplants for a long period, cellular rejection, which appears gradually, must be understood. Two aspects of the problem of cellular rejection must be examined: that of how a transplant recipient's acquired immune system reacts with the transplanted organ; and the problem of runaway porcine immune function, where the porcine T cells and B cells attached to the transplanted organ proliferate in the transplant recipient's body, and recognize the recipient as a foreign object. The RAG-1 gene in miniature swine for xenograft use is knocked out, and post-transplant transfer of the porcine acquired immune system to recipients does not occur in swine without T cells and B cells. As a result, the success rate of xenotransplantation is expected to rapidly increase.

(2) RAG-1 Knockout Swine as Human Organ Incubators for Retrogenerative Medicine

Nude mice lacking a thymus do not possess acquired immunity, due to T cell antigen receptor deficiency, and hence they do not reject human cells and organs. Therefore, transplant organs for plastic surgery, such as human noses and auricles, have been cultured in nude mice. However, mice are limited in size, and larger organs for retrogenerative medicine use cannot be produced.

The body fluid composition and physiological function of omnivorous swine is more similar to humans than that of other herbivorous livestock animals. Swine also have a shorter generation time than primates. Thus, swine are the most suitable animals to use as incubator bodies for culturing human organs for retrogenerative medicine. Since RAG-1 knockout swine have lost their acquired immunity, they are similar to nude mice in that they do not reject human organs, and can supply nutrients and culture organs.

(3) Human Acquired Immunity Developed using Swine

It is predicted that human acquired immunity can be established by transferring human bone marrow into RAG-1 knockout swine, since they do not have their own acquired immunity. If porcine/human bone marrow chimeric animals for acquired immunity can be developed, it will no longer be necessary to humanize mouse antibodies or the like using conventional recombinant DNA techniques. It will be possible to obtain swine that directly produce human antibodies. With regards to T cells, Human and swine MHC (major histocompatibility antigen complex) class II molecules, which have an important role in T cell antigen recognition, are very similar, and human helper T cells that function in humans can therefore be produced. For example, AIDS virus infections are mediated by the CD4 molecule of helper T cells, causing the death of helper T cells. As a result, AIDS-infected patients lose their acquired immunity and die from opportunistic infections. However, CD4 molecules that resist the human AIDS virus have been already constructed. Therefore, acquired immunity can be restored to AIDS patients by making their helper T cells resistant to the AIDS virus, amplifying these cells in RAG-1 knockout swine, and then transferring the cells back into the patients. Immunity and immune activity can be recovered in patients with immunodeficiency, or with reduced immunity due to aging, by injection of their own acquired immune system cells, which have been amplified inside the body of RAG-1 knockout porcine.

1. A porcine-derived DNA of any one of the following (a) to (c):
(a) a DNA comprising the nucleotide sequence of SEQ ID NO: 1 or 2;
(b) a DNA encoding a protein comprising the amino acid sequence of SEQ ID NO: 3;
(c) a DNA encoding a protein comprising an amino acid sequence in which one or more amino acids in the amino acid sequence of SEQ ID NO: 3 have been added, deleted, substituted, and/or inserted, and also comprising a DNA recombination inducing activity.

2. A vector comprising the DNA of claim 1.

3. A transformed cell containing the DNA of claim 1, or the vector of claim 2.

4. A protein encoded by the DNA of claim 1.

5. A method for producing the protein of claim 4, comprising a step of culturing the transformed cell of claim 3, and recovering an expressed protein from the media of the culture.

6. An antibody which binds to the protein of claim 4.

7. A vector comprising a DNA sequence which suppresses the function of endogenous porcine RAG-1 gene by homologous recombination.

8. A porcine cell into which the vector of claim 7 is introduced.

9. A method for producing porcine in which endogenous RAG-1 gene function is suppressed, comprising the following steps (a) and (b):
(a) implanting a nucleus of the cell of claim 8 into an enucleated egg;
(b) implanting the enucleated egg obtained in (a) into a swine.

10. A method for producing a swine with suppressed endogenous RAG-1 gene function, comprising a step of implanting the cell of claim 8 into the swine.
